(19) United States
(12) Patent Application Publication
(10) Pub. No.: US 2014/0072628 A1
(43) Pub. Date: Mar. 13, 2014

(54) STABLE PHARMACEUTICAL COMPOSITION OF SAXAGLIPTIN

(71) Applicants: Atul Kaushik, Navi Mumbai (IN); Arra Ganga Srinivas, Navi Mumbai (IN); Kamal Mehta, Rajasthan (IN)

(72) Inventors: Atul Kaushik, Navi Mumbai (IN); Arra Ganga Srinivas, Navi Mumbai (IN); Kamal Mehta, Rajasthan (IN)

(73) Assignee: Glenmark Generics Ltd., Mahwah, NJ (US)

(21) Appl. No.: 14/023,783

(22) Filed: Sep. 11, 2013

Related U.S. Application Data
(60) Provisional application No. 61/713,322, filed on Oct. 12, 2012.

(30) Foreign Application Priority Data
Sep. 12, 2012 (IN) 2646/MUM/2012

(51) Int. Cl. A61K 9/20; A61K 31/403

(52) U.S. Cl. CPC A61K 9/2004; A61K 31/403
USPC 424/465; 514/412; 424/464; 424/489

(57) ABSTRACT

Disclosed herein is a stable pharmaceutical composition comprising a substrate having deposited on its surface a layer comprising saxagliptin or pharmaceutically acceptable salts thereof, wherein a seal coat is not present between the substrate and the saxagliptin layer.

### PRIORITY

This application claims the benefit under 35 U.S.C. §119 to Indian Provisional Application No. 2646/MUM/2012, filed on Sep. 12, 2012, and to U.S. Provisional Application No. 61/713,322, filed on Oct. 12, 2012, the contents of each of which are incorporated by reference herein.

### FIELD OF INVENTION

The present invention relates to a stable pharmaceutical composition comprising saxagliptin or pharmaceutically acceptable salts thereof

### BACKGROUND OF THE INVENTION

1. Technical Field

Saxagliptin is an orally-active inhibitor of the DPP4 enzyme.

2. Description of the Related Art

U.S. Pat. No. 7,420,079 discloses saxagliptin and its hydrochloride, trifluoroacetic acid and benzoate salts, as well as saxagliptin monohydrate. Saxagliptin is marketed under the trade name ONGLYZA® by Bristol-Myers Squibb for the treatment of type 2 diabetes.

U.S. Pat. No. 6,395,767 discloses saxagliptin, (1S,3S,5S)-2-[(2S)-2-amino-2-(3-hydroxy tricyclo[3.3.1.13,7]dec-1-yl)acetyl]-2-azabicyclo[3.1.0]hexane-3-carbonitrile, its hydrochloride and trifluoroacetic acid salts.

Literature suggests that saxagliptin is susceptible to degradation in pharmaceutical compositions; however, very few attempts have been made to develop a stable pharmaceutical composition of saxagliptin.

U.S. Pat. No. 7,951,400 (US '400) describes saxagliptin undergoes intramolecular cyclization leading to the formation of a degradant known as cyclic amidine (mainly cis-cyclic amidine (CA)), which is therapeutically inactive thus is undesirable. It is believed that the stress generated in regular pharmaceutical operations, and possibly the exposure of saxagliptin to the commonly used pharmaceutical excipients triggers the cyclization, resulting into the formation of cyclic amidine. In US '400, the preferred composition comprises, starting from the center outwards are (a) an inert tablet core (b) inner seal coat (c) saxagliptin layer (d) and a protective coat. The inner seal and outer protective coats of US '400 are believed to avoid the direct contact of saxagliptin with excipients and outside humidity. However, due to the number of coating layers, the compositions of US '400 are prove to be time consuming in terms of manufacturing at a commercial level, with the added challenge of yield optimization.

The present invention provides the preparation of pharmaceutical composition of saxagliptin or its pharmaceutically acceptable salts without any inner seal coat between core tablet and the saxagliptin layer.

### SUMMARY OF THE INVENTION

The present invention provides a stable pharmaceutical composition comprising a substrate having deposited directly on its surface a layer comprising saxagliptin or pharmaceutically acceptable salts thereof and a pharmaceutically acceptable additive.

The present invention provides a stable pharmaceutical composition comprising a substrate having deposited on its surface a layer comprising saxagliptin or pharmaceutically acceptable salts thereof and a pharmaceutically acceptable additive, wherein there is no inner layer between the surface and the layer comprising saxagliptin or pharmaceutically acceptable salts thereof and a pharmaceutically acceptable additive.

In an another embodiment, a stable pharmaceutical composition of present invention comprises a substrate having deposited on its surface a layer comprising saxagliptin or pharmaceutically acceptable salts thereof and a pharmaceutically acceptable additive, wherein the substrate contains acidifying agents.

In one preferred embodiment, a stable pharmaceutical composition of present invention comprising a substrate having deposited on its surface a layer comprising saxagliptin HCl and a pharmaceutically acceptable additive.

In another aspect, the present invention provides a stable pharmaceutical composition comprising saxagliptin HCl, wherein cyclic amidine impurity does not exceed 0.30% when exposed to 60° C. for 7 days.

The present invention provides a stable tablet comprising a tablet core having a layer comprising saxagliptin or pharmaceutically acceptable salts thereof and a pharmaceutically acceptable additive coated directly on its surface.

The present invention provides a stable tablet consisting essentially of a substrate having deposited on its surface a layer comprising saxagliptin or pharmaceutically acceptable salts thereof and a pharmaceutically acceptable additive.

Saxagliptin undergoes an intramolecular cyclization and forms cyclic amidine as degradant which has no therapeutic effect. Cyclic amidine impurity arises due the routine pharmaceutical operations and the excipients used in preparation of pharmaceutical composition of Saxagliptin. Earlier attempts have been made to avoid the contact between commonly used excipients and saxagliptin. One such attempt was made by Desai et. al. in U.S. Pat. No. 7,951,400, wherein the seal coat was applied on a tablet core to avoid the contact between commonly used excipients and saxagliptin. The present invention provides surprisingly a stable pharmaceutical composition of saxagliptin or its pharmaceutically acceptable salt, without any inner seal coat between a layer comprising saxagliptin and the core tablet. The pharmaceutical composition of the present invention advantageously controls the formation of cyclic amidine impurity, even at elevated temperatures.

### DETAILED DESCRIPTION OF INVENTION

It is to be understood that the descriptions of the present invention have been simplified to exemplify the elements that are relevant for the present invention. These elements are not restrictive in their nature as person of ordinary skill in the art will recognize that other elements and/or steps may also be required in executing the present invention. The embodiments exemplified and illustrated herein are for exemplary purposes only, and are not meant to be limited in their description of the present invention.

As used herein, the term “drug” or “active ingredient” or “active” shall refer to saxagliptin, or its pharmaceutically acceptable salts, hydrates or solvates thereof. As used herein, the term “pharmaceutically acceptable salts” shall refer to salts prepared from pharmaceutically acceptable non toxic bases or acids including inorganic or organic bases and inorganic or organic acids, however for the purpose of this invention, saxagliptin hydrochloride (HCl), in form of amorphous, anhydrous or hydrates is preferred. The compositions of the present invention can be prepared by using saxagliptin HCl of amorphous or anhydrous or hydrate nature. Alternatively, the composition of the present invention can also be prepared by using saxagliptin monohydrate or saxagliptin amorphous, wherein saxagliptin monohydrate or saxagliptin amorphous is dissolved in a 0.1N HCl, before incorporating this into the pharmaceutical composition. As used herein, the term “substrate” includes core tablet, water soluble and water insoluble non pareil seeds, mini tablets, beads, beadlet, pellet or spheroids. As used herein, the terms ‘drug layer’ or ‘layer applied on the substrate’ or “a saxagliptin layer” are interchangeable and intended to mean the layer directly applied to the substrate; and as the layer defined above.

In one embodiment, the present invention provides a stable pharmaceutical composition comprising a substrate having deposited directly on its surface a layer of saxagliptin or pharmaceutically acceptable salts thereof and pharmaceutically acceptable additive.

In another embodiment, a stable pharmaceutical composition of the present invention comprises a substrate having deposited directly on its surface a layer comprising saxagliptin hydrochloride (HCl) and a pharmaceutically acceptable additive.

In a preferred embodiment, the present invention provides a stable pharmaceutical composition consisting essentially of a substrate having deposited on its surface a layer saxagliptin HCl and pharmaceutically acceptable additives, wherein the saxagliptin HCl is amorphous or crystalline, preferably a crystalline anhydrous form.

In a further preferred embodiment, the present invention provides the preparation of a stable pharmaceutical composition, the process comprising depositing a coating solution/dispersion comprising saxagliptin HCl and a pharmaceutically acceptable additive over a substrate.

In another preferred embodiment, the present invention provides the preparation of a stable pharmaceutical composition, the process comprising depositing over a substrate a coating solution/dispersion comprising saxagliptin monohydrate dissolved in 0.1 N HCl, and a pharmaceutically acceptable additive.

In one of the aspects, the present invention provides a stable pharmaceutical composition consisting essentially of a substrate having deposited on its surface, a layer comprising saxagliptin or pharmaceutically acceptable salts thereof and a pharmaceutically acceptable additive, wherein the substrate contains a portion of saxagliptin or its pharmaceutically acceptable salts.

In one of the embodiments, the present invention provides a stable pharmaceutical composition comprising a substrate having deposited on its surface a layer comprising saxagliptin or pharmaceutically acceptable salts thereof and a pharmaceutically acceptable additive, wherein the substrate can be a core tablet, water soluble or swellable non pareil seeds, water insoluble non pareil seeds, mini tablets, beads, beadlet, pellet or spheroids. Herein, water soluble or swellable non pareil seeds include, but not limited to, sugar spheres, starch spheres, sugar/starch spheres, microcrystalline cellulose (MCC) spheres. Water insoluble non pareil seed include, but not limited to, silicon dioxide, dicalcium phosphate, calcium stearate, magnesium stearate, glass and ethylcellulose.

In a more preferred embodiment, the present invention provides a stable pharmaceutical composition comprising a substrate having deposited on its surface a layer comprising saxagliptin or pharmaceutically acceptable salts thereof and a pharmaceutically acceptable additive, wherein the substrate is a core tablet.

The core tablet of the present invention can be prepared by wet granulation, dry granulation or roller compaction method. The core tablet of the present invention comprises one or more pharmaceutically acceptable excipients; preferably the core tablet comprises diluents, binders, disintegrants and lubricants. In addition to excipients known in the art, the core tablet may additionally contain an acidifying agent, which maintains the acidic pH of the core. The present invention provides the preparation of the core tablet, the process comprising granulating with water or 0.1N HCl as a granulating solvent using wet granulation method; preferably with 0.1N HCl.

In another embodiment, the present invention provides the preparation of the core, the process comprising direct compression or dry granulation method, wherein when employing either method, an acidifying agent should be incorporated in the core. More preferably, the present invention provides the preparation of the core tablet by wet granulation.

In one of the preferred embodiments, the present invention provides a stable pharmaceutical composition comprising a substrate having deposited on its surface, a layer comprising saxagliptin or pharmaceutically acceptable salts thereof and pharmaceutically acceptable additive, wherein a pharmaceutically acceptable additive is selected from the group including, but not limited to, the polymers, waxy substances, readily available coating dispersions.

In one of the preferred embodiment, a stable pharmaceutical composition consisting essentially of a substrate having deposited on its surface a layer comprising saxagliptin or pharmaceutically acceptable salts thereof and at least one pharmaceutically acceptable additive wherein pharmaceutically acceptable additive is a water soluble polymer, water insoluble polymer or combinations thereof.

Exemplary polymers to be used in a layer containing the drug are polyvinylpyrrolidone (PVP), polyalkylene glycol such as polyethylene glycol, gelatin, polyvinyl alcohol (PVA), starch and derivatives thereof, cellulose derivatives, such as hydroxypropylmethyl cellulose (HPMC), hydroxypropylcellulose, carboxymethylcellulose, methyl cellulose, ethyl cellulose, hydroxyethylcellulose, carboxyethylcellulose, carboxymethylhydroxyethylcellulose, acrylic acid polymers, polymethacrylates, or any other pharmaceutically acceptable polymer. Preferably for the purpose of the present invention polyvinyl alcohol or HPMC alone or in combination are preferred choices of polymers.

Apart from the pharmaceutically acceptable additives mentioned above, saxagliptin layer (a drug layer) may optionally contain plasticizers, opacifiers and colorants. Herein, the pharmaceutically acceptable additives further comprise the readily available coating dispersions such as, Opadry, Opadry II that are based on polymers such as PVA or HPMC. Readily available coating dispersions such as, Opadry, Opadry II generally contain pharmaceutically acceptable additives such as PVA or HPMC, plasticizers, opacifiers and optional colorants.

In the preferred embodiment, the present invention provides a stable pharmaceutical composition consisting essentially of a core tablet having deposited directly on its surface a layer comprising saxagliptin HCl and a pharmaceutically acceptable additive. Furthermore, the present invention provides a stable pharmaceutical composition consisting essentially of a core tablet having deposited directly on its surface a layer comprising saxagliptin HCl and polyvinylalcohol. In an alternative aspect, the present invention presents a pharmaceutical composition consisting essentially of a core tablet having deposited directly on its surface a layer comprising saxagliptin HCl and hydroxypropylmethyl cellulose (HPMC).

In one preferred embodiment, the present invention provides a stable pharmaceutical composition comprising a core tablet having deposited directly on its surface a layer comprising saxagliptin HCl, polyvinyl alcohol, a plasticizer, an opacifier and optionally colorants. Alternatively, the present invention provides a stable pharmaceutical composition comprising a core tablet having deposited directly on its surface a layer comprising saxagliptin HCl, HPMC, a plasticizer, an opacifiers and optionally colorants.

The present invention provides a stable pharmaceutical composition comprising about 80-90% by weight of the substrate, and about 10-20% by weight of a layer comprising saxagliptin HCl and pharmaceutically acceptable additive, to the total weight of composition. A stable pharmaceutical composition of the present invention comprises about 80-90% by weight of the substrate, and about 10-20% by weight of a layer comprising saxagliptin HCl and PVA, to the total weight of composition. A stable pharmaceutical composition of the present invention comprises about 80-90% by weight of the substrate, and about 10-20% by weight of a layer comprising saxagliptin HCl and HPMC, to the total weight of composition.

A stable pharmaceutical composition of present invention comprises, about 80-90% by weight of the core tablet and 10-20% by weight of a layer comprising saxagliptin HCl, polyvinyl alcohol and plasticizers, to the total weight of composition. However in a more specific aspect, a stable pharmaceutical composition of present invention comprises, about 80-90% by weight of the core tablet and 10-20% by weight of a layer comprising saxagliptin HCl, polyvinyl alcohol, plasticizers, opacifiers and colorants, to the total weight of composition.

A stable pharmaceutical composition of present invention comprises, about 80-90% by weight of the core tablet and 10-20% by weight of a layer comprising saxagliptin HCl, HPMC and plasticizers, to the total weight of composition. A pharmaceutical composition of present invention may comprise, about 80-90% by weight of the core tablet and 10-20% by weight of a layer comprising saxagliptin HCl, HPMC, plasticizers, opacifiers and colorants, to the total weight of composition.

In a more preferred embodiment, a stable pharmaceutical composition of the present invention comprises about 80-90% by weight of the core tablet, and a drug layer containing about 1-5% of saxagliptin HCl, about 1-6% of polyvinyl alcohol, 0.5-3.0% of plasticizers, 0.2-5% opacifiers to the total weight of composition. The stable pharmaceutical composition of the present invention comprises saxagliptin HCl and polyvinyl alcohol/HPMC in ratio of 1:1 to 1:5.

Though it is not essential but the stable pharmaceutical composition of saxagliptin or its pharmaceutically acceptable salts may optionally contain an outer coating layered over the saxagliptin layer (a drug layer). The said outer coating may contain the pharmaceutically acceptable additive, same as that of a drug layer, or alternatively the outer coat may employ a pharmaceutically acceptable additive that is different than the one used in drug layer. The outer coat of present invention smoothen the surface of a composition, prevents the abrasion of the drug layer during the regular handling and packaging of a composition. The outer coat of present invention contains pharmaceutically acceptable additive, plasticizers, opacifiers and optionally colorant. Readily available coating dispersions such as, but not limited to, Opadry, Opadry II may also be used, to coat the outer coat, which generally contains a pharmaceutically acceptable additives such as PVA or HPMC, plasticizers, opacifiers and optionally colorant.

The polymers used in an outer coating include, but not limited to polyvinylpyrrolidone (PVP), polyalkylene glycol such as polyethylene glycol, gelatin, polyvinyl alcohol (PVA), starch and derivatives thereof, cellulose derivatives, such as hydroxypropylmethyl cellulose (HPMC), hydroxypropyl cellulose, carboxymethyl cellulose, methyl cellulose, ethyl cellulose, hydroxyethyl cellulose, carboxyethylcellulose, carboxymethylhydroxyethyl cellulose, acrylic acid polymers, polymethacrylates, or any other pharmaceutically acceptable polymer.

In another embodiment, a stable pharmaceutical composition of the present invention comprises, (a) a substrate having deposited on its surface, a layer comprising saxagliptin HCl, and a pharmaceutically acceptable additive and (b) an outer coating.

In another embodiment, a stable pharmaceutical composition of present invention is prepared by (a) depositing a coating solution/dispersion comprising saxagliptin monohydrate dissolved in 0.1 N HCl, and a pharmaceutically acceptable additive, over a substrate, (b) further depositing an outer coating comprising a pharmaceutically acceptable additive.

In another preferred embodiment, a stable pharmaceutical composition of the present invention comprises (a) a core tablet having deposited on its surface, a layer comprising saxagliptin HCl and PVA and (b) an outer coating. Alternatively in another preferred embodiment, a stable pharmaceutical composition of the present invention comprises (a) a core tablet having deposited on its surface, a layer comprising saxagliptin HCl and HPMC and (b) an outer coating.

In another embodiment, a stable pharmaceutical composition of the present invention comprises (a) a substrate containing about 0.5-5% be weight total saxagliptin HCl, having deposited on its surface, a layer comprising rest of 95-99.5% of saxagliptin HCl, and a pharmaceutically acceptable additive and (b) an outer coating.

A stable pharmaceutical composition of the present invention comprises, (a) a core tablet having deposited on its surface, a layer comprising saxagliptin HCl, a PVA, a plasticizer, a opacifiers and optionally colorants and (b) an outer coating containing a polymer, a plasticizer and optionally opacifiers and colorants.

A stable pharmaceutical composition of the present invention comprises, about 80-90% by weight of a core tablet, about 10-15% by weight of a layer comprising saxagliptin HCl and a pharmaceutically acceptable additive and about 2-7% by weight of outer coating, to the total weight of composition. Preferably a stable pharmaceutical composition of the present invention comprises, about 80-90% by weight of a core tablet, about 10-15% by weight of a layer comprising saxagliptin HCl, PVA and about 2-7% by weight of outer coating having PVA, to the total weight of composition. More preferably a stable pharmaceutical composition of present invention comprises, about 80-90% by weight of a core tablet, about 10-15% by weight of a layer comprising saxagliptin HCl, PVA, a plasticizer, an opacifier, and about 2-7% by weight of outer coating having PVA, a plasticizer, an opacifier, to the total weight of composition.

A stable pharmaceutical composition of the present invention comprises, about 80-90% by weight of a core tablet, about 10-15% by weight of a layer comprising saxagliptin HCl, HPMC and about 2-7% by weight of outer coating having HPMC, to the total weight of composition. Preferably a stable pharmaceutical composition of the present invention comprises, about 80-90% by weight of a core tablet, about 10-15% by weight of a layer comprising saxagliptin HCl, HPMC, a plasticizer, an opacifier, and about 2-7% by weight of outer coating having HPMC, a plasticizer, an opacifier, to the total weight of composition.

The core tablet of present invention comprises about 150-180 mg of diluent: 15-30 mg of disintegrants; 5-10 mg of binders; 1-5 mg of glidant and/or lubricants and optionally an acidifying agent.

In one of the preferred embodiments, stable composition comprising a substrate having deposited on its surface, a layer comprising saxagliptin HCl and at least one pharmaceutically acceptable additive, wherein total cyclic amidine impurity does not exceed the acceptable range during accelerated stability conditions. More particularly, the present invention provides a pharmaceutical composition of saxagliptin or its pharmaceutically acceptable salts wherein the total cyclic amidine impurity is not more than about 0.30% when exposed at 60° C. for 7 days in closed conditions. The present invention provides pharmaceutical compositions which are evaluated for % total cyclic amidine impurity, when exposed to conditions of 25° C./60% RH, 30° C./65% RH and 40° C./75% RH conditions.

It is not expressly required for the success of the invention, but the pharmaceutical composition of the present invention can be packed in a container containing an oxygen scavenger such as stabilox canisters. The present invention also provides pharmaceutical composition comprising saxagliptin HCl optionally packed in a container having an additional oxygen scavenger stabilox canister, wherein an unknown impurity does not exceed more than about 0.1%.

The tableting powder contains a number of inert materials known as excipients. They may be classified according to the role they play in the tablet. The primary composition includes fillers, binders or diluents, lubricants, disintegrants, acidifying agents and glidants. Other excipients which give physical characteristics to the finished tablet are coloring agents, and flavors in the case of chewable tablets. Typically, excipients are added to a formulation to impart good flow and compression characteristics to the material being compressed. Such properties are imparted to these excipients through pretreatment steps, such as wet granulation, slugging, spray drying etc.

One or more fillers or diluents can be selected. Examples of pharmaceutically acceptable fillers or diluents include, but are not limited to sucrose, and lactose, in particular lactose monohydrate, mannitol, sorbitol, microcrystalline cellulose, powdered cellulose. Different grades of lactose or microcrystalline cellulose can be used. In case of a water soluble active ingredient, like the one described in this invention, more preferably water insoluble fillers, such as starch, microcrystalline cellulose, dibasic calcium phosphate dihydrate, and anhydrous dibasic calcium phosphate, preferably microcrystalline cellulose, can be used in addition or instead of the water soluble fillers. In a preferred embodiment, a combination of water soluble and water insoluble fillers are used. Preferably, a combination of lactose and microcrystalline cellulose is the preferred diluent. The total weight percentage of filler ranges between about 65% and about 90% by weight, preferably from about 70% to about 80%.

Disintegrants are often included to ensure that the tablet has an acceptable rate of disintegration. One or more disintegrants can be selected. Examples of pharmaceutically acceptable disintegrants include, but are not limited to starches; clays; celluloses; alginates; gums; cross-linked polymers, e.g., crospovidone, cross-linked Ca-CMC and Na-CMC, Sodium starch glycolate. Disintegrants are included to ensure that the tablet has an acceptable rate of disintegration. Croscarmellose sodium is the preferred disintegrant for this formulation. Preferably the disintegrant is present in the tablet formulation in an amount of from about 1% to about 12% by weight.

Another commonly used class of excipients in tablets is binders. Binders are agents, which impart cohesive qualities to the powdered material. The compositions described herein also can comprise binders, examples of pharmaceutically acceptable binders include, but are not limited to povidones (e.g., PVP K-30, PVP K-60, and PVP K-90), cellulose derivatives (e.g., methylcellulose and sodium carboxymethylcellulose), gelatin, polyethylene glycol, polymethacrylates, ethyl cellulose, hydroxypropylcellulose, hydroxypropylmethylcellulose, hydroxyethylcellulose, pregelatinized starch and sodium alginate, wherein HPMC is particularly preferred. The total weight percentage of binder ranges between about 1% and about 5% by weight. Most preferred range is about 2-2.5%.

Lubricants are typically added to prevent the tableting materials from sticking to punches, minimize friction during tablet compression, and allow for removal of the compressed tablet from the die. Examples of pharmaceutically acceptable lubricants include, but are not limited to, magnesium stearate, calcium stearate, canola oil, glyceryl palmitostearate, hydrogenated vegetable oil, magnesium oxide, mineral oil, poloxamer, polyethylene glycol, polyvinyl alcohol, sodium benzoate, sodium lauryl sulfate, sodium stearylfumarate, stearic acid, talc, hydrogenated vegetable oil, zinc stearate and the like. In one embodiment, magnesium stearate is included as a lubricant in an amount of about 0.2% to about 2.0%, preferably about 1%, by weight of the tablet.

Acidifying agents are typically added to the composition to lower the pH of the composition, while acting as stabilizers. Examples of pharmaceutically acceptable lubricants include, but are not limited to, ascorbic acid, tartaric acid, malic acid, cysteine HCl, citric acid. In one embodiment cysteine HCl is included as an acidifying agent in an amount of about 0.2% to about 2.0%, preferably about 1%, by weight of the tablet.

In an another aspect, the present invention provides a process for preparing a stable pharmaceutical composition of saxagliptin HCl, the process comprising, preparing a substrate; (a) preparing a solution or suspension having saxagliptin HCl and pharmaceutically acceptable additives; (b) coating the solution or suspension containing saxagliptin HCl and pharmaceutically acceptable additives, onto the substrate; (c) drying the coated tablet; optionally, providing an outer coating over the drug coated substrate.

In another aspect, the present invention provides the preparation of the drug layer (or saxagliptin layer), the process comprising dissolving saxagliptin monohydrate in 0.1N HCl under continuous stirring; adding to this, pharmaceutically acceptable additives, and stirring to the formation of a uniform suspension.

The following examples describe compositions of the present invention containing saxagliptin or its pharmaceutically acceptable salts, but they are not to be interpreted as limiting the scope of the claims.

### Example 1

Composition having substrate loaded with drug coating along with PVA based Opadry. The ingredients and amounts are set forth below in Table 1.

TABLE I

| | |
|---|---|
| Ingredient | Qty/Tab (in mg) |

CORE TABLET

| | |
|---|---|
| Lactose Monohydrate | 71.00 |
| Microcrystalline Cellulose | 96.00 |
| Croscarmellose Sodium | 25.00 |
| Hydroxy propyl methyl cellulose (E5 LV) | 6.00 |
| 0.1N HCl | qs |
| Magnesium stearate | 2.00 |
| Total | 200.00 |

DRUG LOADING

| | |
|---|---|
| Saxagliptin monohydrate | 5.28 |
| Opadry white 85F18422 (PVA based) | 20.00 |
| 0.1N HCl | qs |
| Total | 225.28 |

The manufacturing process consists of following:

(A) Lactose monohydrate, microcrystalline cellulose, croscarmellose sodium and HPMC were co-sifted and added to a high speed mixer granulator and mixed for a desired time. Dry mix was granulated with 0.1 N HCl in water. Granules were dried until the desired loss on drying (LOD) was achieved. Drying was followed by lubrication with Mg-stearate for sufficient time. Lubricated granules were compressed on suitable punches.

(B) Drug Suspension: Saxagliptin monohydrate was dissolved in 0.1N HCl under stirring until a clear solution was obtained. Opadry white 85F18422 was added to the same drug solution under stirring until a uniform suspension was obtained. The pH of the suspension was kept below 3.

(C) Drug loading: Core tablets of (A) were loaded on coating pan and preheated and then coated with suspension of (B) until the desired weight was achieved.

### Example 2

Composition having substrate loaded with drug coating along with PVA based Opadry followed by outer coating using same PVA based opadry. The ingredients and amounts are set forth below in Table 2.

TABLE 2

| | |
|---|---|
| Ingredient | Qty/Tab (in mg) |

CORE TABLET

| | |
|---|---|
| Lactose Monohydrate | 71.00 |
| Microcrystalline Cellulose | 96.00 |
| Croscarmellose Sodium | 25.00 |
| Hydroxy propyl methyl cellulose (E5 LV) | 6.00 |
| 0.1N HCl | qs |
| Magnesium stearate | 2.00 |
| Total | 200.00 |

DRUG LOADING

| | |
|---|---|
| Saxagliptin monohydrate | 5.28 |
| Opadry white 85F18422 (PVA based) | 20.00 |
| 0.1N HCl | qs |

FILM COATING/OUTER COATING

| | |
|---|---|
| Opadry white 85F18422 (PVA based) | 12.00 |
| 0.1N HCl | qs |
| Total | 237.28 |

The manufacturing process consists of following:

(A) Lactose monohydrate, microcrystalline cellulose, croscarmellose sodium and HPMC were co-sifted and added to a high speed mixer granulator and mixed for a desired time. Dry mix was granulated with 0.1N HCl in water. Granules were dried until the desired LOD was achieved. Drying was followed by lubrication with Mg-stearate for sufficient time. Lubricated granules were compressed on suitable punches.

(B) Drug Suspension: Saxagliptin monohydrate was dissolved in 0.1 N HCl under stirring until a clear solution was obtained. Opadry white 85F18422 was added to the same drug solution under stirring until a uniform suspension was obtained. The pH of the suspension was kept below 3.

(C) Drug loading: core tablets of (A) were loaded on coating pan and preheated and then coated with suspension of (B) until the desired weight was achieved.

(D) Film coating/Outer coating: Opadry white 85F18422 was dispersed in 0.1N HCl under constant stirring until a uniform suspension obtained. Coated the drug loaded tablets with this suspension until the desired weight was achieved.

### Example 3

Composition having substrate loaded with drug coating along with PVA based Opadry followed by outer coating using Opadry II pink. The ingredients and amounts are set forth below in Table 3.

TABLE 3

| | |
|---|---|
| Ingredient | Qty/Tab (in mg) |

CORE TABLET

| | |
|---|---|
| Lactose Monohydrate | 71.00 |
| Microcrystalline Cellulose | 96.00 |
| Croscarmellose Sodium | 25.00 |
| Hydroxy propyl methyl cellulose (E5 LV) | 6.00 |
| 0.1N HCl | qs |
| Magnesium stearate | 2.00 |
| Total | 200.00 |

DRUG LOADING

| | |
|---|---|
| Saxagliptin monohydrate | 5.28 |
| Opadry white 85F18422 (PVA based) | 20.00 |
| 0.1N HCl | qs |

FILM COATING/OUTER COATING

| | |
|---|---|
| Opadry II Pink 85F540094 (PVA based) | 12.00 |
| 0.1N HCl | qs |
| Total | 237.28 |

The manufacturing process consists of following:

(A) Lactose monohydrate, microcrystalline cellulose, croscarmellose sodium and HPMC were co-sifted and added to a high speed mixer granulator and mixed for a desired time. Dry mix was granulated with 0.1N HCl in water. Granules were dried until the desired LOD was achieved. Drying was followed by lubrication with Mg-stearate for sufficient time. Lubricated granules were compressed on suitable punches.

(B) Drug Suspension: Saxagliptin monohydrate was dissolved in 0.1N HCl under stirring till clear solution was obtained. Opadry white 85F18422 was added to the same drug solution under stirring till uniform suspension obtained. pH of the suspension was kept below 3.

(C) Drug loading: core tablets of (A) were loaded on a coating pan and preheated and then coated with suspension of (B) until the desired weight was achieved.

(D) Film coating/Outer coating: Opadry II pink 85F540094 was dispersed in 0.1 N HCl under constant stirring until the uniform suspension was obtained. Coated the drug loaded tablets with this suspension until the desired weight was achieved.

### Example 4

Composition having substrate loaded with drug coating along with HPMC based opadry followed by outer coating with HPMC and opacifier. The ingredients and amounts are set forth below in Table 4.

TABLE 4

| | |
|---|---|
| Ingredient | Qty/Tab (in mg) |

CORE TABLET

| | |
|---|---|
| Lactose Monohydrate | 71.00 |
| Microcrystalline Cellulose | 96.00 |
| Croscarmellose Sodium | 25.00 |
| Hydroxy propyl methyl cellulose (E5 LV) | 6.00 |
| 0.1N HCl | qs |
| Magnesium stearate | 2.00 |
| Total | 200.00 |

DRUG LOADING

| | |
|---|---|
| Saxagliptin monohydrate | 5.28 |
| Opadry white 03F580015 (HPMC based) | 20.00 |
| 0.1N HCl | qs |

FILM COATING/PROTECTIVE COATING

| | |
|---|---|
| Hydroxypropyl methyl cellulose 3 cps | 4.35 |
| Polyethylene Glycol 6000 | 1.05 |
| Titanium Dioxide | 0.6 |
| 0.1N HCl | qs |
| Total | 231.28 |

The manufacturing process consists of following:

(A) Lactose monohydrate, microcrystalline cellulose, croscarmellose sodium and HPMC were co sifted and added to high speed mixer granulator and mixed for desired time. Dry mix was granulated with 0.1N HCl in water. Granules were dried until the desired LOD was achieved. Drying was followed by lubrication with Mg-stearate for sufficient time. Lubricated granules were compressed on suitable punches.

(B) Drug Suspension: Saxagliptin monohydrate was dissolved in 0.1 N HCl under stirring until a clear solution was obtained. Opadry white 03F580015 was added to the same drug solution under stirring until a uniform suspension was obtained. The pH of the suspension was kept below 3.

(C) Drug loading: Core tablets of (A) were loaded on coating pan and preheated and then coated with suspension of (B) until the desired weight was achieved.

(D) Film coating: HPMC, PEG and titanium dioxide were dispersed in 0.1N HCl under constant stirring until a uniform suspension was obtained. Coated the drug loaded tablets with this suspension until a desired weight was achieved.

### Example 5

Composition having substrate loaded with drug coating along with HPMC based opadry followed by outer coating with HPMC and opacifier and colorant. The ingredients and amounts are set forth below in Table 1.

TABLE 5

| | |
|---|---|
| Ingredient | Qty/Tab (in mg) |

CORE TABLET

| | |
|---|---|
| Lactose Monohydrate | 71.00 |
| Microcrystalline Cellulose | 96.00 |
| Croscarmellose Sodium | 25.00 |
| Hydroxy propyl methyl cellulose (E5 LV) | 6.00 |
| 0.1N HCl | qs |
| Magnesium stearate | 2.00 |
| Total | 200.00 |

DRUG LOADING

| | |
|---|---|
| Saxagliptin monohydrate | 5.28 |
| Opadry white 03F580015 (HPMC based) | 20.00 |
| 0.1N HCl | qs |

FILM COATING/PROTECTIVE COATING

| | |
|---|---|
| Hydroxypropyl methyl cellulose 3 cps | 4.32 |
| Polyethylene Glycol 6000 | 1.05 |
| Titanium Dioxide | 0.6 |
| Iron oxide red | 0.03 |
| 0.1N HCl | qs |
| Total | 231.31 |

The manufacturing process consists of following:

(A) Lactose monohydrate, microcrystalline cellulose, croscarmellose sodium and HPMC were co-sifted and added to a high speed mixer granulator and mixed for a desired time. Dry mix was granulated with 0.1N HCl in water. Granules were dried until the desired LOD was achieved. Drying was followed by lubrication with Mg-stearate for sufficient time. Lubricated granules were compressed on suitable punches.

(B) Drug Suspension: Saxagliptin monohydrate was dissolved in 0.1N HCl under stirring until a clear solution was obtained. Opadry white 03F580015 was added to the same drug solution under stirring until a uniform suspension was obtained. The pH of the suspension was kept below 3.

(C) Drug loading: core tablets of (A) were loaded on coating pan and preheated and then coated with suspension of (B) until the desired weight was achieved.

(D) Film coating: HPMC, PEG, titanium dioxide and iron oxide were dispersed in 0.1N HCl under constant stirring until a uniform suspension was obtained. Coated the drug loaded tablets with this suspension till desired weight was achieved.

### Example 6

Composition having substrate loaded with drug coating of Saxagliptin HCl along with PVA based Opadry. The ingredients and amounts are set forth below in Table 6.

TABLE 6

| | |
|---|---|
| Ingredient | Qty/Tab (in mg) |

CORE TABLET

| | |
|---|---|
| Lactose Monohydrate | 71.00 |
| Microcrystalline Cellulose | 96.00 |
| Croscarmellose Sodium | 25.00 |
| Hydroxy propyl methyl cellulose (E5 LV) | 6.00 |
| 0.1N HCl | qs |
| Magnesium stearate | 2.00 |
| Total | 200.00 |

DRUG LOADING

| | |
|---|---|
| Saxagliptin HCl | 5.58 |
| Opadry white 85F18422 (PVA based) | 20.00 |
| purified water | qs |
| Total | 225.58 |

The manufacturing process consists of following:

(A) Lactose monohydrate, microcrystalline cellulose, croscarmellose sodium and HPMC were co-sifted and added to a high speed mixer granulator and mixed for a desired time. Dry mix was granulated with 0.1N HCl in water. Granules were dried until the desired LOD was achieved. Drying was followed by lubrication with Mg-stearate for sufficient time. Lubricated granules were compressed on suitable punches.

Drug Suspension: Saxagliptin HCl was dissolved in water under stirring until a clear solution was obtained. Opadry white 85F18422 was added to the same drug solution under stirring until a uniform suspension obtained.

Drug loading: core tablets of (A) were loaded on a coating pan and preheated and then coated with suspension of (B) until the desired weight was achieved.

Stability Data

Pharmaceutical composition of the present invention prepared according to above given methods were subjected to 60° C. for 7 days in HDPE containers without desiccant and without void filler to monitor the level of total cyclic amidine in it.

Table 7 below summarizes the level of total cyclic amidine produced in these compositions during a three day and seven day period at 60° C.

TABLE 7

| | % total cyclic amidine impurity | | |
|---|---|---|---|
| Examples | Initial | 3 days at 60° C. | 7 days at 60° C. |
| Example 1 | 0.06 | 0.07 | 0.07 |
| Example 2 | 0.04 | 0.06 | 0.14 |
| Example 3 | 0.04 | 0.07 | 0.07 |
| Example 4 | 0.04 | 0.09 | 0.28 |
| Example 5 | 0.05 | 0.11 | 0.24 |
| Onglyza ® 5 mg | 0.06 | 0.08 | 0.07 |

The table above shows that the compositions having no inner seal coat between the core tablet and the drug layer has comparable levels of % of total cyclic amidine impurity to those found in the commercially available Onglyza®.

Pharmaceutical composition of the present invention prepared according to above given methods were subjected to 25° C./60% RH and 40° C./75% RH for 3M in HDPE containers with desiccant and cotton to monitor the level of total cyclic amidine in it. Table 8 set forth below summarizes the level of total cyclic amidine produced in these compositions during 3 months period at different stability conditions.

TABLE 8

| | % Impurity | | | | | | | | |
|---|---|---|---|---|---|---|---|---|---|
| | Initial | | | 3M 25° C./60% RH | | | 3M 40° C./75% RH | | |
| | | % Highest | | | % Highest | | | % Highest | |
| | Cyclic | unknown | % Total | Cyclic | unknown | % Total | Cyclic | unknown | % Total |
| Impurities | amidine | impurity | impurity | amidine | impurity | impurity | amidine | impurity | impurity |
| Onglyza 5 mg | 0.01 | 0.02 | 0.12 | — | — | — | 0.07 | 0.04 | 0.23 |
| Onglyza 2.5 mg | 0.06 | 0.10 | 0.18 | — | — | — | 0.09 | 0.03 | 0.20 |
| 5 mg | 0.05 | ND* | 0.05 | 0.04 | 0.01 | 0.08 | 0.15 | ND* | 0.16 |
| 2.5 mg | 0.05 | ND* | 0.05 | 0.05 | 0.03 | ND* | 0.14 | ND* | 0.14 |

*Not detected

From the above table, it can be concluded that compositions having no inner seal coat in between the substrate and the drug layer have comparable level % of impurity with Onglyza®.

What is claimed is:

1. A stable pharmaceutical composition comprising a substrate having deposited on its surface a layer comprising saxagliptin or pharmaceutically acceptable salts thereof, wherein a seal coat is not present between the substrate and the saxagliptin layer.

2. The stable pharmaceutical composition of claim 1, wherein the layer of saxagliptin further contains one or more pharmaceutically acceptable additives.

3. The stable pharmaceutical composition of claim 2, wherein the one or more pharmaceutically acceptable additives are selected from the group consisting of polymers, waxy substances, and readily available coating dispersions.

4. The stable pharmaceutical composition of claim 2, wherein the one or more pharmaceutically acceptable additives are a polyvinyl alcohol or a hydroxypropylmethyl cellulose based coating dispersion.

5. The stable pharmaceutical composition of claim 1, wherein the substrate is a core tablet, water soluble and water insoluble non pareil seeds, mini tablets, beads, beadlet, pellet or spheroids.

6. The stable pharmaceutical composition of claim 1, wherein the substrate is a core tablet.

7. The stable pharmaceutical composition of claim 1, wherein saxagliptin is incorporated as a monohydrate or amorphous, and converts to the hydrochloride salt in the process of depositing over a substrate.

8. The stable pharmaceutical composition of claim 7, wherein saxagliptin hydrochloride is amorphous or crystalline.

9. The stable pharmaceutical composition of claim 1, wherein total cyclic amidine impurity doesn't exceed 0.15% at 3M 40° C./75% RH stability condition.

10. A process for preparing a stable pharmaceutical composition of saxagliptin, the process comprising:
(a) preparing a substrate;
(b) preparing a solution or suspension having saxagliptin HCl and pharmaceutically acceptable additives;
(c) coating the solution or suspension containing saxagliptin HCl and pharmaceutically acceptable additives upon the substrate; and
(d) drying the coated tablet.
